SEQUENCE LISTING (6 sequences)

SEQ ID NO: 1 - DNA, 18 bp, synthetic construct
  source 1..18: mol_type = other DNA
gccatctcctgatgacgc

SEQ ID NO: 2 - DNA, 18 bp, synthetic construct
  source 1..18: mol_type = other DNA
atttaccgcagccagacg

SEQ ID NO: 3 - DNA, 40 bp, synthetic construct
  source 1..40: mol_type = other DNA
ctggggcgaggtcgtggtattccgacaaacaccacgaatt

SEQ ID NO: 4 - DNA, 42 bp, synthetic construct
  source 1..42: mol_type = other DNA
cattttgcagctgtacgctcgcagcccatcatgaatgttgct

SEQ ID NO: 5 - DNA, 24 bp, synthetic construct
  source 1..24: mol_type = other DNA
ctttgtaacaacctgtcatcgaca

SEQ ID NO: 6 - DNA, 25 bp, synthetic construct
  source 1..25: mol_type = other DNA
atcaatctcgatatccatgaaggtg

(19) United States
(12) Patent Application Publication
(10) Pub. No.: US 2026/0103764 A1
(43) Pub. Date: Apr. 16, 2026

(54) Method and Device for Detecting Multiple Foodborne Pathogens

(71) Applicants: University of Cincinnati, Cincinnati, OH (US); National Yang Ming Chiao Tung University, Hsinchu City, Taiwan (CN)

(72) Inventors: Jiajie Diao, Lebanon, OH (US); Chen-Yi Lee, Hsinchu (TW)

(73) Assignee: University of Cincinnati, Cincinnati, OH (US)

(21) Appl. No.: 19/116,750

(22) PCT Filed: Sep. 28, 2023
(86) PCT No.: PCT/US2023/034059

§ 371 Date: Mar. 28, 2025

Related U.S. Application Data
(60) Provisional application No. 63/410,660, filed on Sep. 28, 2022.

(51) Int. Cl. C12Q 1/689; C12Q 1/6806

(52) U.S. Cl. CPC C12Q 1/689; C12Q 1/6806

(57) ABSTRACT

A method for detecting multiple pathogens is provided. The method involves linking multiple pathogens to fluorophores and then obtaining emission spectra of the pathogens using a prism-based fluorescence imaging system. In one embodiment, emission spectra of the fluorophores are obtained using optical detection and at least one other aspect of the pathogens is obtained using a silicon chip.

### CROSS-REFERENCE TO RELATED APPLICATIONS

This application is a continuation of PCT Application No. PCT/US23/34059 filed Sep. 28, 2023, which claims the benefit of the filing date of U.S. Provisional Application No. 63/410,660 filed Sep. 28, 2022, the disclosure of which is incorporated by reference herein in its entirety.

### FIELD OF THE INVENTION

This invention relates generally to methods for detecting foodborne pathogens.

### BACKGROUND OF THE INVENTION

This section is intended to introduce the reader to various aspects of the art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Foodborne pathogens have been the cause of many diseases in the U.S. (United States) and worldwide. Although researchers have identified more than 250 types of foodborne illnesses, the CDC (Centers for Disease Control and Prevention) estimates that each year 3,000 people die and 1 in 6 Americans get foodborne illness. The World Health Organization (WHO) estimates that 600 million people fall ill after consuming contaminated food and beverages every year. In the U.S., the cumulative financial costs of foodborne illnesses are estimated at $15.6 billion each year by the U.S. Department of Agriculture. The most common bacteria and viruses that cause foodborne illness in the U.S. are Norovirus, Salmonella, Clostridium perfringens, Campylobacter, and Staphylococcus aureus (Staph). For example, Salmonella is commonly associated with food and water borne infections leading to gastrointestinal diseases. This causes a major economic impact, so early detection is crucial. Other germs don't cause as many illnesses, but when they do, the illnesses are more likely to lead to hospitalization. Examples of these germs include Clostridium botulinum (botulism), Listeria, Escherichia coli (E. coli), and Vibrio.

In general, detection and diagnostics initially relied on culture-based methods and immunoassays and have progressed to using molecular biology-based methods such as polymerase chain reaction (PCR). The aim has always been to find a rapid, sensitive, specific, and cost-effective method. The major advantages of PCR are the speed and sensitivity of the process. This can be an alternative to the tedious time-consuming procedure of culturing and identifying of pathogens in food safety laboratories. However, the use of PCR for pathogens still requires the development of better methods to overcome certain disadvantages, such as cell lysis, nucleic acid extraction, cross-contamination, or failed reaction. These disadvantages can lead to inconsistent results and reduce the appeal of PCR as a reliable approach.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a method for detecting multiple pathogens is provided. The method involves linking multiple pathogens to fluorophores and then obtaining emission spectra of the pathogens using a prism-based fluorescence imaging system. In one embodiment, emission spectra of the fluorophores are obtained using optical detection and at least one other aspect of the pathogens is obtained using a silicon chip. In one embodiment, the at least one other aspect of the pathogens is selected from the group consisting of movement, coalescence, separation, RNA extraction, DNA extraction, and heating cycle. In another embodiment, at least four spectra are distinguished using relative intensities of the fluorophores observed in different spectral windows.

In one embodiment, a nano-droplet comprising the multiple pathogens is merged with four different colors of fluorophores. In another embodiment, multiple nano-droplets comprising the multiple pathogens are used in a multiplex polymerase chain reaction (PCR) test. In one embodiment, at least five nano-droplets are used in a multiplex PCR test.

In another embodiment of the invention, a method for detecting one or more pathogens is provided. The method involves combining a sample containing the one or more pathogens with a Loop-Mediated Isothermal Amplification (LAMP) solution to form a mixture, applying the mixture to a biochip, heating the biochip and observing changes in the samples using a microscope, wherein the LAMP solution comprises at least four primers designed to target a specific pathogen.

In one embodiment, the biochip is heated at a temperature of at least about 65° C. for at least 30 minutes. In another embodiment, the specific pathogen is E. coli. In one embodiment, the primers target the malB gene. In another embodiment, the primers are four different primers comprising either SEQ. 1, SEQ. 2, SEQ. 3 or SEQ. 4. In one embodiment, the LAMP solution comprises at least six primers. In another embodiment, the primers are six different primers comprising either SEQ. 1, SEQ. 2, SEQ. 3, SEQ. 4, SEQ, 5 or SEQ. 6.

### BRIEF DESCRIPTION OF THE DRAWINGS

The objects and advantages of the present invention will be further appreciated in light of the following detailed descriptions and drawings in which:

FIG. 1A is a graph showing the excitation spectra of fluorophores linked with specific pathogens 1-4.

FIG. 1B is a graph showing the emission spectra of fluorophores linked with specific pathogens 1-4. The excitation light can be at 532 nm.

FIG. 2 is a schematic showing a microfluidic operation for mixing samples with reagent.

FIG. 3 is a pair of images showing DNA or RNA extraction using magnetic particles.

FIG. 4 is a pair of images showing PCR thermal cycling.

FIG. 5A is an image showing the setup of the biochip test platform.

FIG. 5B is an image showing a PC-MEDA biochip and micro-photo of the testing area.

FIG. 5C is an image showing a thermal image from an infrared camera of the biochip under heating mode (Bx1 was selected as the whole testing area).

FIG. 5D is a graph showing the temperature vs time curve of the biochip from 0 s to 600 s when LAMP test mode start.

FIG. 6A is an image showing the agarose gel results of an E. coli LAMP test. L: 1 kb DNA Ladder. N4: negative control of four primers LAMP. N6: negative control of six primers LAMP. P4: positive sample of four primers LAMP. P6: positive sample of six primers LAMP. Microscope images of E. coli LAMP test on biochip at 0 min and 30 min.

FIG. 6B is a series of images showing negative control samples for the LAMP test of FIG. 6A.

FIG. 6C is a series of images showing positive samples for the LAMP test of FIG. 6A.

FIG. 7A is a series of images showing negative samples from real-time photos of samples on biochip during the four primers LAMP reaction.

FIG. 7B is a series of images showing negative samples from real-time photos of samples on biochip during the four primers LAMP reaction.

FIG. 7C is a series of images showing positive samples from real-time photos of samples on biochip during the four primers LAMP reaction.

FIG. 7D is a series of images showing positive samples from real-time photos of samples on biochip during the four primers LAMP reaction.

FIG. 8 is an image showing the agarose gel results of a sensitivity test for LAMP test using four primers. The lanes labeled as 1-7 correspond to sample 1-sample 7 in Table 1. L: 1 kb DNA Ladder. N: negative control.

### DEFINITIONS

As used herein, the term “about,” when referring to a value or to an amount of mass, weight, time, volume, pH, size, concentration, or percentage, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

### DETAILED DESCRIPTION OF THE INVENTION

One or more specific embodiments of the present invention are described below. In an effort to provide a concise description of these embodiments, all features of an actual implementation may not necessarily be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Early stage and rapid detection of pathogen contamination are needed to prevent the large-scale outbreak of diseases. Pathogen contamination can occur during the production, processing, and/or preparation of food. Also, pathogens can come from a polluted source, or seafood captured from the water that was polluted. The devices of the present invention can address many problematic aspects of field testing for food safety, agriculture safety, water safety, self-diagnosis, and disease monitoring.

In one embodiment, the present invention uses a bio-field programmable gate array for polymerase chain reaction detection of multiple foodborne pathogens. This novel device is sensitive, reliable, and utilizes a multiplex polymerase chain reaction (PCR) test based on multiple droplets for the manipulation of microfluidic operations. The device is implemented using standard CMOS technology to perform all the functions required for PCR, including temperature control, heating, microfluidics, and fluorescence detection. The device is autonomous, portable, and reliable while handling numerous fluidic functions like calibrated volume dispensing, sub-volume fragmentations, coalescence, mixing, and reagent storage. Combined with optical detection, the device, in conjunction with a silicon-chip, can detect multiple DNA or RNA sequences to distinguish 20 different types of pathogens simultaneously. The initial sample only requires 10 nanoliters, which is much smaller than traditional PCR methods. The silicon chip is capable of handling numerous fluidic functions, including calibrated volume dispensing, sub-volume fragmentations, movement, coalescence, mixing, DNA or RNA extraction, and thermal cycling.

### Fluorophores

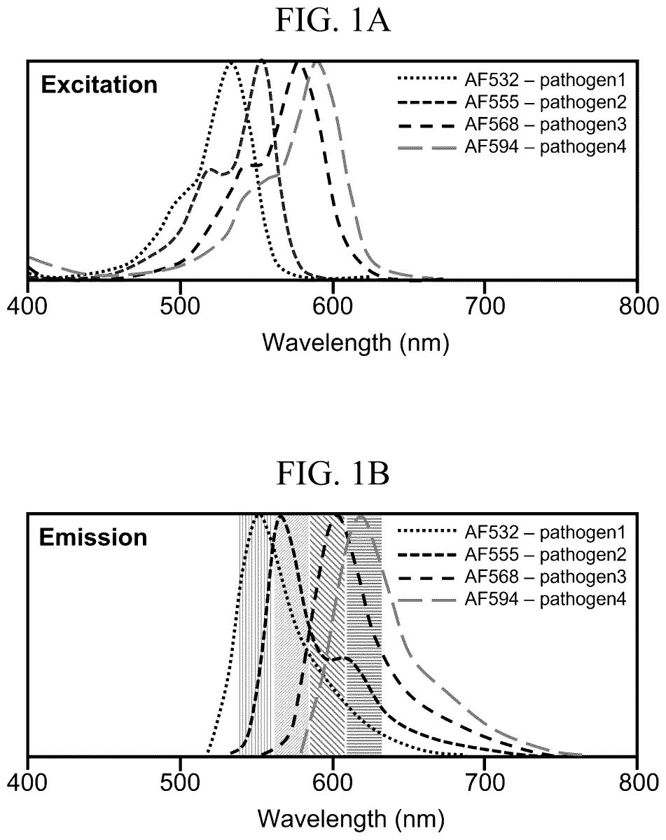

Proper fluorophores should be selected for use in the present invention, allowing for detection of 20 pathogens simultaneously using optical detection. In FIGS. 1A and 1B, a four-color system of fluorophores using the same light irradiation is illustrated. The fluorophores were Alexa Fluor (AF) 532, AF555, AF568, AF594, etc. A novel aspect of the device of the present invention is its ability to distinguish four spectra by the relative intensities observed in different spectral windows, via a prism-based fluorescence imaging scheme to obtain the emission spectra in the field of view. An important element of prism-based fluorescence imaging is that it doesn't require any specific filter while also providing the ratio of intensity at the different wavelength. To detect 20 pathogens simultaneously, the sample can be prepared to have five nano-droplets for use in PCR cycling at the same time. Each nano-droplet is merged with four different colors of fluorophores, and then moved to the detection region to identify pathogens from each sample.

### Biochip System

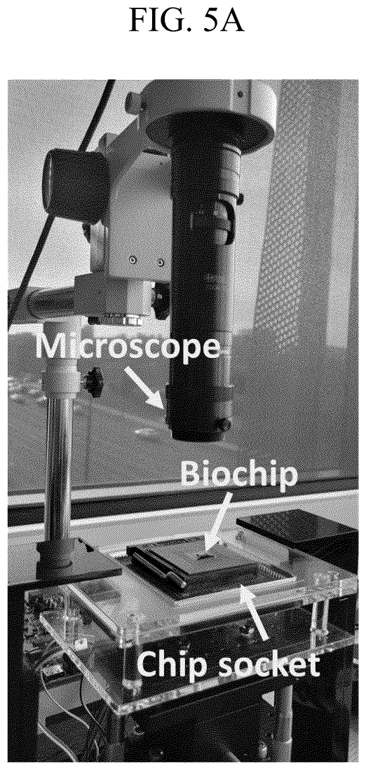

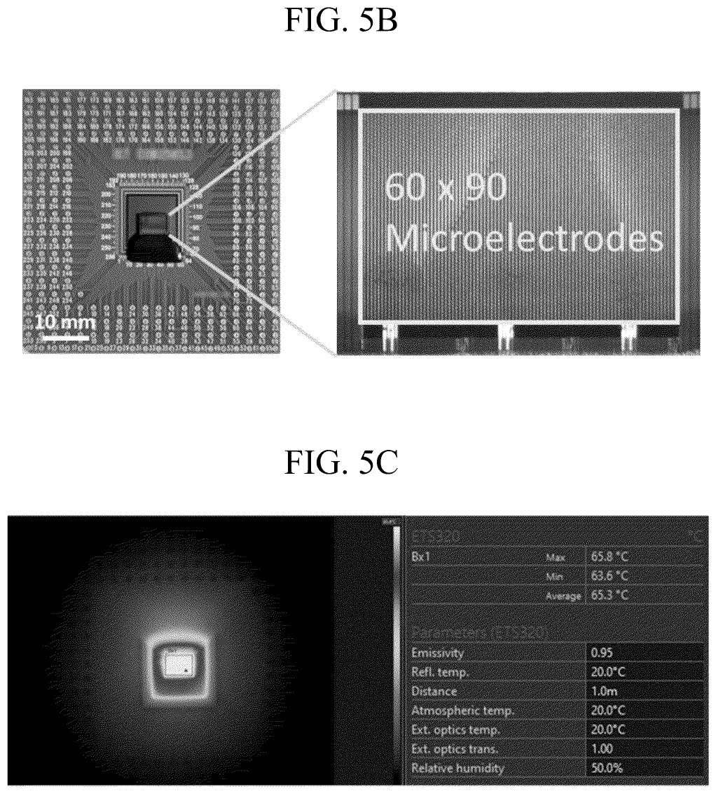

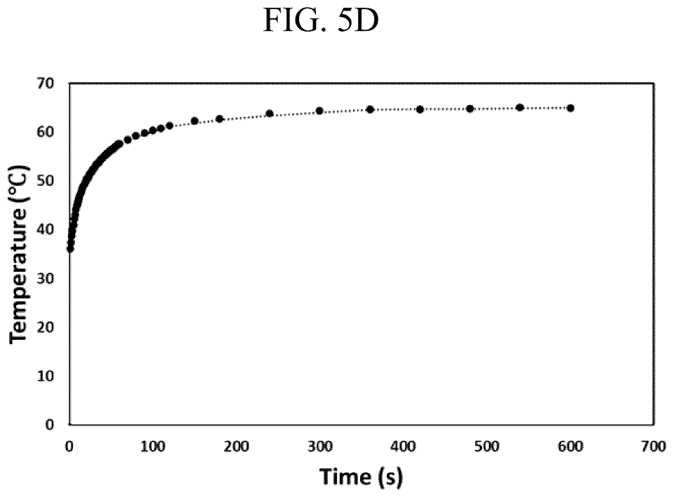

Another embodiment of the present invention uses a biochip test platform. An embodiment of the chip used is shown in FIGS. 5A-5C. The chip is capable of offering the voltage to manipulate the droplet, including movement to separate, mixing, DNA or RNA extraction, and heating cycle for PCR. The FIG. 5A-5C demonstrated the heating cycle and temperature measurement using IR camera. An IR camera was used to measure LAMP mode temperature during presetting and the adjusting stage. The heating curve was recorded (see FIG. 5D).

### LAMP Results

A four primer Loop-Mediated Isothermal Amplification (“LAMP”) and six primer LAMP were used to detect E. coli in water through a biochip test platform. Loop primer can improve the efficiency of amplification reaction. The six primer LAMP provides good test results. The four primer LAMP can also give an accurate test result, so it is still a good choice for the lower cost.

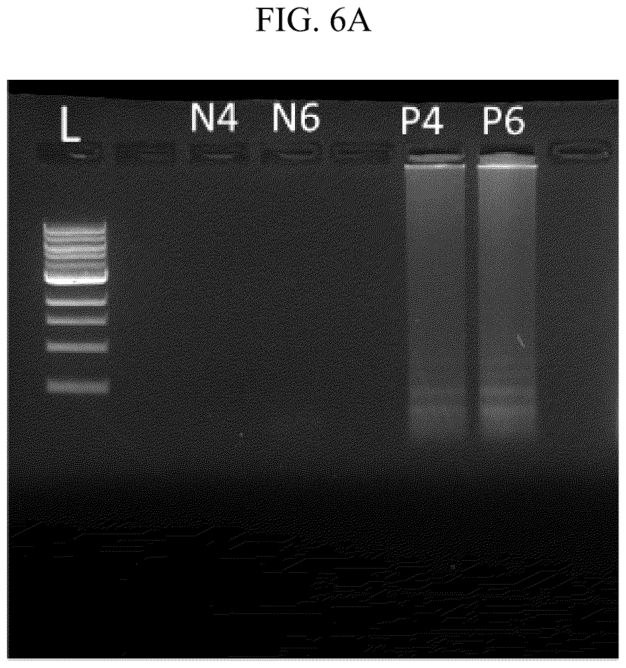

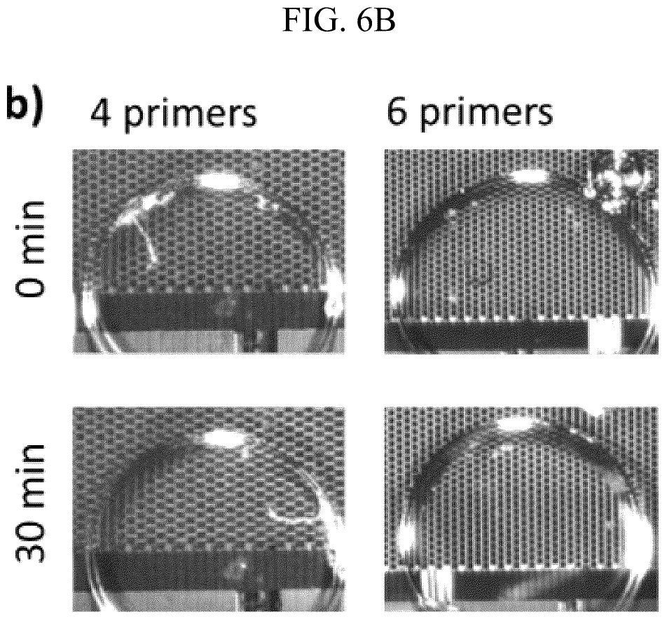

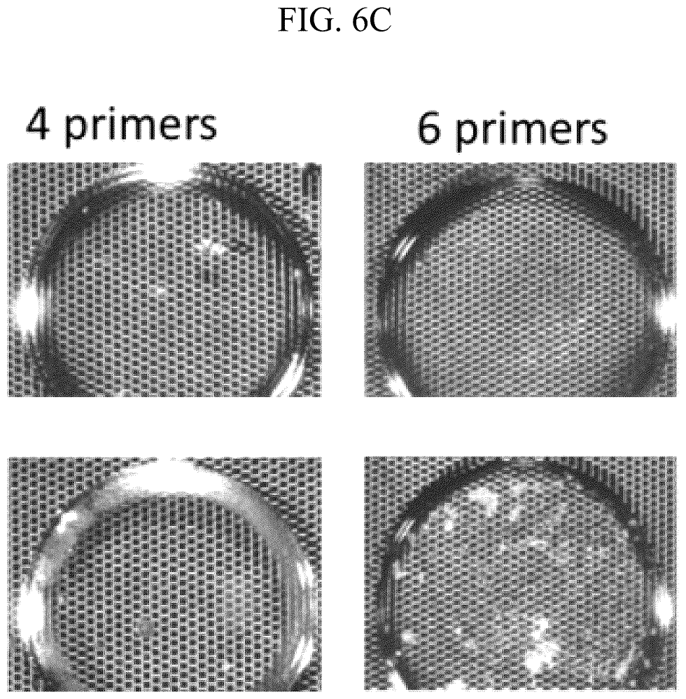

The tests verified that the two kinds of primer mix can work for E. coli detection. FIGS. 6B and 6C show four primer LAMP and six primer LAMP test results through traditional in tube heating (see FIG. 6C) as well as the innovative biochip LAMP mode of the present invention (see FIG. 6B). FIG. 6A is the end-point agarose gel result of negative and positive samples. Positive samples show a bright ladder like pattern while negative samples had no signal. FIG. 6B is the microscope image of a LAMP sample on chip. All sample droplets were transparent at the start, and negative samples remained transparent after 30 min. Positive samples produced white precipitate due to the amplification. These results prove that both primer mixes can give an accurate test result of an E. coli sample, and this detection process can be done on a biochip test platform.

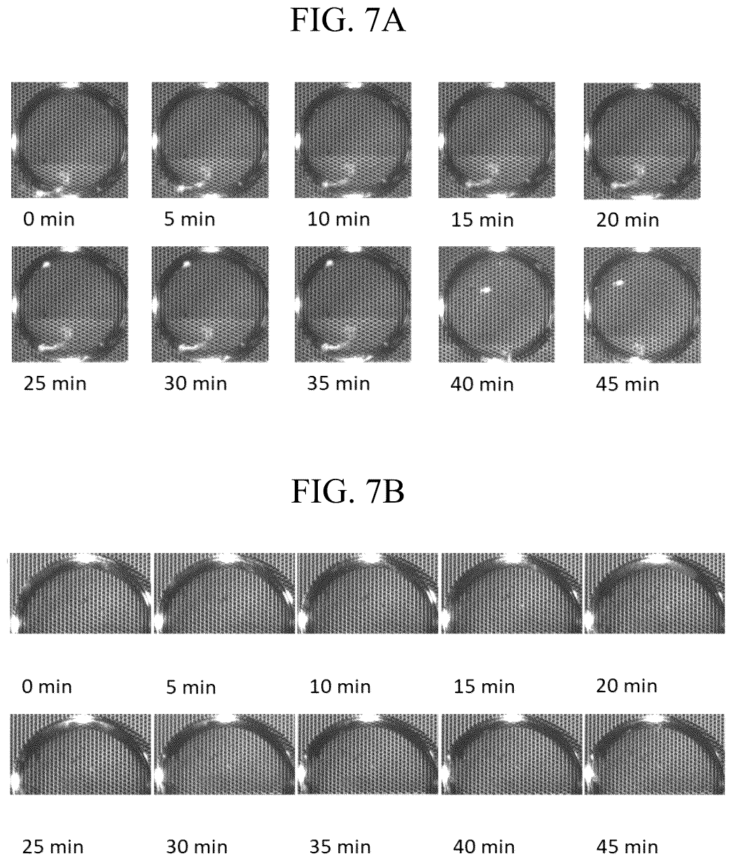

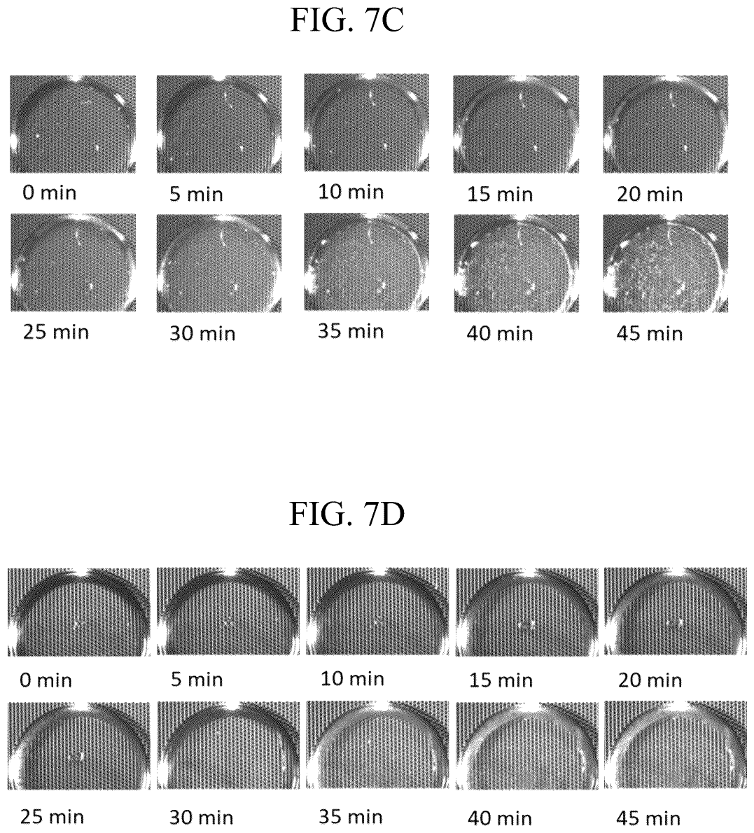

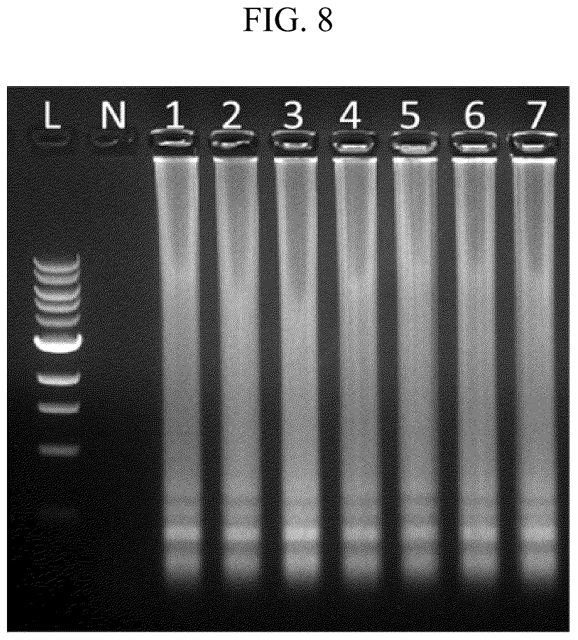

In the traditional test method, a LAMP sample is kept in tubes with a lid and heated on a heating block. After reaction, the sample needs to be taken out of the tube to run gels. This step has a risk of pollution, given the high concentration of gene copies after exponential amplification. And the agarose gel, loading buffer and electrophoresis instrument need to be prepared. Our biochip system can provide one-stop test with real-time imaging. After loading samples, the LAMP test mode is started. The electrode array on the chip can reach the testing temperature in a minute and the whole reaction process can be observed and recorded by a microscope camera. This enables the use of naked eyes to distinguish positive samples. FIGS. 7A-7D show a series of real-time tracking images during the LAMP test.

The sensitivity of LAMP was determined as shown in FIG. 8. By using the LAMP reaction, E. coli samples can be detected successfully at concentrations as low as 10 copies/μL.

### EXAMPLES

### Example 1: PCR-Based Test

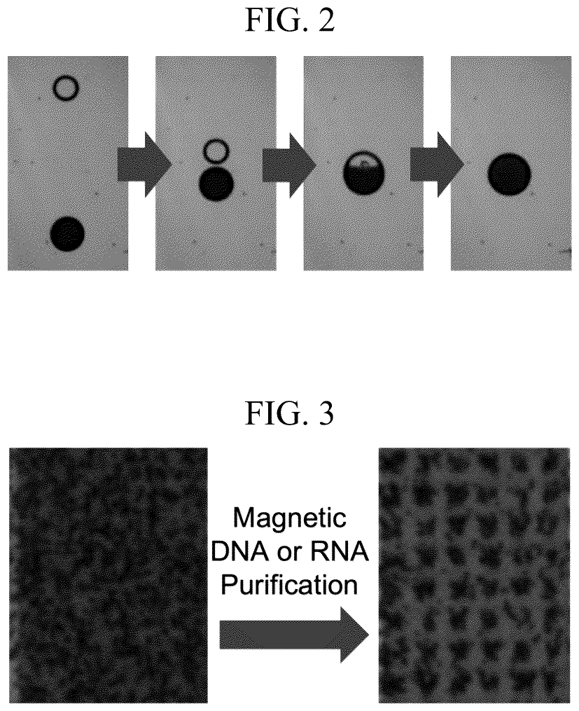

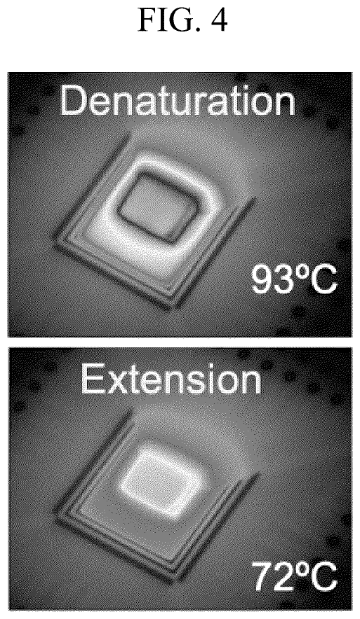

Twenty samples were prepared and simultaneously detected using the following method:
1) Sample delivery—Samples and reagents are delivered to the target regions.
2) Adjust sample concentration—Samples (or reagent) are cut or mixed and the droplets are moved to certain regions, where capacitive sensing can be exploited to confirm both location and volume. Note that the operation of cutting or mixing can be repeated. (See FIG. 2).
3) DNA or RNA extraction—Magnetic particles are specifically functionalized to allow for quick and efficient purification directly after their extraction from samples extracts (see FIG. 3). Centrifugation steps were avoided.
4) Fast-thermal—cycling PCR-Temperature is controlled by a device to provide various reactions as defined in the PCR protocol (see FIG. 4). Also, real-time quantification of amplicons can be performed using the optical platform.
5) Optical sensing—All of the samples (droplets) are covered by silicone oil. This enables fast and easy analysis for an extremely wide dynamic range of quantification and significantly high reliability and sensitivity. The fluorescent signals are collected throughout the PCR process (monitorization of the process of amplification in real time using fluorescence) not only at the end of the reaction. The results can be used to quantify the initial amounts of pathogens with high precision over a wide range of concentration.

### Example 2: Biochip-Based Test

### Materials

2× WarmStart LAMP Master Mix (WarmStart LAMP Kit (DNA & RNA), #E1700S, New England Biolabs), Ethidium bromide stock solution (VWR), Molecular Biology Grade Water (Fisher Scientific), 1 kb DNA ladder (#N3232S, New England Biolabs).

### LAMP Primers

E. coli-specific primers were used that targeted malB gene. These primers are in the E. coli GenBank sequence (GDB J01648). The malB gene is conserved in E. coli lineage and is not shared with other gram-negative bacteria. Primers were designed based on the study of Hill et al. and shown in the “Sequences” section. Both four primers and six primers were tested. Primers F3, B3, FIP and BIP comprised the four-primer system. Primers F3, B3, FIP, BIP, Loop F and Loop B comprised the six-primer system.

A 10× four primer mix was made with 16 μM FIP, 16 μM BIP, 2 μM F3, 2 μM B3 in water, while a 10× six primer mix contained 16 μM FIP, 16 μM BIP, 2 μM F3, 2 μM B3, 4 μM Loop F, 4 μM Loop B in water. The concentration of each primer in the 25 μL LAMP reaction mix was 0.2 μM F3 and B3 primers, 1.6 μM FIP and BIP primers, 0.4 μM Loop F and Loop B.

### Samples Collection

E. coli (BL21 strain) was used to evaluate the specificity and sensitivity of the LAMP reaction and grown in Luria-Bertani (LB) broth medium. E. coli bacterial pellets were collected by centrifuging live culture E. coli at 1200 rpm for 3 min at room temperature with a swing-bucket rotor centrifuge. The pellets were re-suspended in nuclease-free water in microcentrifuge tubes and heated at 95° C. for 10 min. The mixture was centrifuged at 10000 rpm for 10 min with microcentrifuge and supernatant was collected. The sample was frozen at −20° C. before using.

### Dilute Sample Preparation

10-fold serial dilutions of an E. coli sample were prepared for testing the sensitivity of the LAMP detection. DNA concentration was measured by NanoDrop 1000 Spectrophotometer (Thermo Fisher Scientific Inc.) DNA copy number was calculated through the following equation:

$$DNA c opy number = \frac{c \times 1 {0}^{- 9} g \times 6.0221 \times 1 {0}^{23} molecules / mol}{5 \times 1 {0}^{6} bp \times 650 g / mol} \qquad Eq . 1$$

Where c is the concentration of DNA, 6.0221×1023 is Avogadro's constant, 5×106 bp is the length of E. coli gene, and 650 is the average mass of 1 bp DNA.

The expected copy numbers for each E. coli sample are shown in Table 1. The tested concentration and calculated copy number of original E. coli sample and first three dilute samples are also enclosed while other samples are too dilute to be measured.

TABLE 1

| | | | Calculation | Dilution ratio |
|---|---|---|---|---|
| | Number of | DNA | number | (Dilute |
| | DNA copy | concentration | of DNA | from the |
| Samples | (copies/μL) | (ng/μL) | copy | last sample) |
| Original | N/A | 218.5 | 4.04870 × 10⁷ | N/A |
| 1 | 1 × 10⁷ | 56.26666667 | 1.04260 × 10⁷ | 1:4 |
| 2 | 1 × 10⁶ | 5.666666667 | 1.05001 × 10⁶ | 1:10 |
| 3 | 1 × 10⁵ | 0.566666667 | 1.05001 × 10⁵ | 1:10 |
| 4 | 1 × 10⁴ | N/A | N/A | 1:10 |
| 5 | 1 × 10³ | N/A | N/A | 1:10 |
| 6 | 1 × 10² | N/A | N/A | 1:10 |
| 7 | 1 × 10¹ | N/A | N/A | 1:10 |

### LAMP Reagents

A mix of 12.5 μL 2× LAMP Master Mix, 2.5 μL 10× Primer Mix, and 2 μL E coli sample was used as the positive sample, whereas the same volume of nuclease-free water was used for the negative control. The testing solution was filled with nuclease-free water until a final volume of 25 μL was obtained.

### LAMP Test

To initiate the experiment, a volume of 1.5 μL from the mixed LAMP solution was applied onto the biochips, which were then covered with Indium tin oxide (ITO) glass. The droplet on the biochips was surrounded by silicon oil. The remaining 23.5 μL of the solution was placed in polymerase chain reaction (PCR) tubes for further processing. The biochips were subjected to a temperature of 65° C. for a duration of 30 min in the testing area. Throughout the procedure, the microscope was employed to observe and track the entire process, which was also recorded using video. For the remaining sample in the PCR tubes, a Programmable Thermal Controller PTC-100 (MJ Research Inc.) was utilized. The incubation temperature was set to 65° C., and the tubes were incubated for 30 min.

To assess the end-point results and detect nucleic acid amplification in the tube samples, a 1% agarose gel containing 0.5 ug/mL ethidium bromide (EB) was employed.

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

SEQUENCES
Outer forward and backward primers:
F3-SEQ. ID 1: 5′-GCCATCTCCTGATGACGC-3′
B3-SEQ. ID 2: 5′-ATTTACCGCAGCCAGACG-3′
Inner forward and backward primers
FIP-SEQ. ID 3:
5′-CTGGGGCGAGGTCGTGGTAT-TCCGACAAACACCACGAATT-3′
BIP-SEQ. ID 4:
5′-CATTTTGCAGCTGTACGCTCGC-AGCCCATCATGAATGTTGCT-3′
Loop forward and backward primers
Loop F-SEQ. ID 5: CTTTGTAACAACCTGTCATCGACA
Loop B-SEQ. ID 6: ATCAATCTCGATATCCATGAAGGTG

What is claimed is:

1. A method of detecting multiple pathogens, the method comprising linking multiple pathogens to fluorophores and then obtaining emission spectra of the pathogens using a prism-based fluorescence imaging system.

2. The method of claim 1 wherein emission spectra of the fluorophores are obtained using optical detection and at least one other aspect of the pathogens is obtained using a silicon chip.

3. The method of claim 1 wherein the at least one other aspect of the pathogens is selected from the group consisting of movement, coalescence, separation, RNA extraction, DNA extraction, and heating cycle.

4. The method of claim 1 wherein at least four spectra are distinguished using relative intensities of the fluorophores observed in different spectral windows.

5. The method of claim 4 wherein a nano-droplet comprising the multiple pathogens is merged with four different colors of fluorophores.

6. The method of claim 5 wherein multiple nano-droplets comprising the multiple pathogens are used in a multiplex polymerase chain reaction (PCR) test.

7. The method of claim 6 wherein at least five nano-droplets are used in a multiplex PCR test.

8. A method of detecting one or more pathogens, the method comprising combining a sample containing the one or more pathogens with a Loop-Mediated Isothermal Amplification (LAMP) solution to form a mixture, applying the mixture to a biochip, heating the biochip and observing changes in the samples using a microscope, wherein the LAMP solution comprises at least four primers designed to target a specific pathogen.

9. The method of claim 8 wherein the biochip is heated at a temperature of at least about 65° C. for at least 30 minutes.

10. The method of claim 8 wherein the specific pathogen is E. coli.

11. The method of claim 10 herein the primers target the malB gene.

12. The method of claim 10 wherein the primers are four different primers comprising either SEQ. 1, SEQ. 2, SEQ. 3 or SEQ. 4.

13. The method of claim 8 wherein the LAMP solution comprises at least six primers.

14. The method of claim 13 wherein the primers are six different primers comprising cither SEQ. 1. SEQ. 2. SEQ. 3. SEQ. 4. SEQ. 5 or SEQ. 6.
